(19) United States
(12) Patent Application Publication
(10) Pub. No.: US 2013/0172837 A1
(43) Pub. Date: Jul. 4, 2013

(54) Negative Pressure Wound Therapy Apparatus Including a Fluid Line Coupling

(71) Applicants: Gregory Kenny, Wakefield, MA (US); John DePiano, JR., Burlington, MA (US); James L. Carlyon, Farmington, MO (US); David E. Fink, Franklin, MA (US); Richard M. Braga, North Easton, MA (US)

(72) Inventors: Gregory Kenny, Wakefield, MA (US); John DePiano, JR., Burlington, MA (US); James L. Carlyon, Farmington, MO (US); David E. Fink, Franklin, MA (US); Richard M. Braga, North Easton, MA (US)

(73) Assignee: TYCO HEALTHCARE GROUP LP, Mansfield, MA (US)

(21) Appl. No.: 13/608,610

(22) Filed: Sep. 10, 2012

Related U.S. Application Data
(63) Continuation of application No. 12/437,069, filed on May 7, 2009, now abandoned.
(60) Provisional application No. 61/051,825, filed on May 9, 2008.
(60) Provisional application No. 61/094,527, filed on Sep. 5, 2008.

(51) Int. Cl. A61M 1/00

(52) U.S. Cl. CPC A61M 1/0031
USPC 604/319

(57) ABSTRACT

A negative pressure wound therapy apparatus includes a wound dressing for forming a substantially fluid-tight seal over a wound to define a reservoir over the wound in which a negative pressure may be maintained. A vacuum source is in fluid communication with the reservoir to provide an appropriate negative pressure to the reservoir to stimulate healing of the wound. The apparatus includes a fluid conduit in fluid communication with the reservoir and defining a fluid flow path for carrying fluids from the reservoir, and a fluid line coupling adapted for selective engagement and disengagement of first and second coupling segments to respectively maintain and interrupt the fluid flow path.

### CROSS-REFERENCE TO RELATED APPLICATIONS

This application claims priority to, and the benefit of, U.S. Provisional Patent Application No. 61/051,825, filed on May 9, 2008 and U.S. Provisional Patent Application No. 61/094,527, filed on Sep. 5, 2008, the entire contents of which are incorporated herein by reference.

### BACKGROUND

1. Technical Field

The present disclosure relates generally to an apparatus for stimulating healing of a wound by applying a reduced pressure over the wound. In particular, the disclosure relates to an apparatus including a fluid line coupling to facilitate disconnection of apparatus components.

2. Background of Related Art

One technique that has proven effective in promoting the healing of wounds is known as negative pressure wound therapy (NPWT). Application of a negative pressure, e.g. reduced or sub-atmospheric pressure, to a localized reservoir over a wound has been found to assist in closing the wound by promoting blood flow to the area, stimulating the formation of granulation tissue and encouraging the migration of healthy tissue over the wound. A negative pressure may also inhibit bacterial growth by drawing fluids from the wound such as exudates, which may tend to harbor bacteria. This technique has proven particularly effective for chronic or healing-resistant wounds, and is also used for other purposes such as post-operative wound care.

The general NPWT protocol provides for a wound to be covered to facilitate suction at the wound area. For example, a flexible membrane having an adhesive periphery might be used to form a substantially fluid-tight seal around a perimeter of the wound, thus providing a reservoir over the wound where a negative pressure may be maintained. A fluid conduit may include a vacuum tube introduced into the reservoir through the membrane to provide fluid communication to an external vacuum source. Atmospheric gas, wound exudates or other fluids may thus be drawn from the reservoir through the fluid conduit to stimulate healing of the wound. Exudates drawn from the reservoir may be deposited in a collection canister until they may be conveniently emptied or the canister replaced.

There is often a need to disconnect various components from the fluid conduit of an NPWT apparatus during an NPWT treatment. For example, replacement of the collection canister may be required at any such time as the canister is filled to capacity. Because fluids may continue to flow through the fluid conduit throughout an NPWT treatment, changing the canister may reduce the effectiveness of the treatment by requiring an interruption in the treatment. Accordingly, a need exists for an NPWT apparatus that permits disconnection of individual components of the apparatus, such as the collection canister, and optionally without disruption of the NPWT treatment. Generally, negative pressure wound therapy provides for a wound to be covered to facilitate suction at the wound area. A conduit is introduced through the wound covering to provide fluid communication to an external vacuum source. Atmospheric gas, wound exudates, or other fluids may thus be drawn from the reservoir through the fluid conduit to stimulate healing of the wound. Exudates drawn from the reservoir may be deposited in a container. Unfortunately, the exudates removed from the wound may be offensive both visually and with respect to smell. This may prove troublesome with an ambulatory system for wound therapy where the removed exudates may be viewed in the tubing or collection canister.

### SUMMARY

Accordingly, the present disclosure is directed to a negative pressure wound therapy apparatus. The apparatus includes a wound dressing for defining a reservoir over a wound in which a negative pressure may be maintained by forming a substantially fluid-tight seal around the wound, a fluid conduit in fluid communication with the reservoir and defining a fluid flow path for carrying fluids from the reservoir, a vacuum source in fluid communication with the fluid conduit and suitable for providing an appropriate negative pressure to the reservoir to stimulate healing of the wound and a fluid line coupling in line with the fluid conduit. The fluid line coupling includes first and second coupling segments adapted for selectable engagement and disengagement therebetween to respectively maintain and interrupt the fluid flow path of the conduit. At least one of the first and second coupling segments is adapted for obstructing the flow of fluid there through upon disengagement from the other of the first and second coupling segments.

The fluid conduit may include a first fluid conduit section and a second fluid conduit section. The first fluid conduit section is coupled to the first coupling segment of the fluid line coupling, and the second fluid conduit section is coupled to the second coupling segment of the fluid line coupling such that fluid may flow between the first and second fluid conduit sections when the first and second coupling segments are engaged and fluid flow is obstructed when the first and second coupling segments are disengaged. The apparatus may further comprise a collection canister coupled to the fluid conduit.

In one embodiment, the at least one of the first and second coupling segments capable of obstructing fluid flow may include a post having a bias to deform the fluid conduit section coupled thereto upon disengagement from the other of the first and second coupling segments to obstruct fluid flow. The other of the first and second coupling segments may include a pin adapted to engage the post when first and second coupling segments are engaged such that the post is moved against the bias to allow the fluid conduit section to assume an un-deformed shape to permit fluid flow there through.

In embodiments, the at least one of the first and second coupling segments capable of obstructing fluid flow may include a plug having a bias to engage sealing surfaces on the coupling segment upon disengagement from the other of the first and second coupling segments to obstruct fluid flow. The other of the first and second coupling segments may include a pin adapted to engage the plug when first and second coupling segments are engaged such that the plug is moved away from the sealing surfaces against the bias to permit fluid flow between the first and second coupling segments.

Alternatively, the at least one of the first and second coupling segments capable of obstructing fluid flow includes a flipper door having a rotational bias to engage sealing surfaces on the coupling segment upon disengagement from the other of the first and second coupling segments to obstruct fluid flow. Alternatively, the at least one of the first and second coupling segments capable of obstructing fluid flow may include a duckbill valve.

The fluid line coupling may include a locking mechanism adapted to move the fluid line coupling between a locked configuration wherein the first and second coupling segments resist separation and an un-locked configuration wherein the first and second mating may be separated. The locking mechanism may include a locking ring adapted to move interfering members on the first coupling segment between a radially inward position to capture a protrusion on the second coupling segment and radially outward position to release the protrusion. Alternatively, the locking mechanism may include a bayonet lock. In another alternative, the locking mechanism includes a flexure arm on the first coupling segment. The flexure arm may have a catch adapted to engage a notch on the second coupling segment when the first and second coupling segments are in the locked configuration.

In an embodiment, at least one of the first and second coupling segments includes a vacuum port having a flange adapted to facilitate connection of the at least one the first and second coupling segments to a wound dressing. The vacuum port defines a fluid flow path therethrough and is adapted for selectable engagement and disengagement with the fluid conduit to respectively maintain and interrupt the fluid flow path through the vacuum port.

In another embodiment, at least one of the first and second coupling segments includes a cantilevered locking tab having a locking feature for engaging a corresponding locking feature on the other of the first and second coupling segments. The locking feature is positioned on the locking tab between a control surface at a free end of the locking tab and a cantilevered support end of the locking tab. The first coupling segment may include at least one opening therein, and the second coupling segment may include a locking tab having a free end configured to protrude laterally through the opening in the first coupling segment to an exterior of the fluid line coupling. The locking tab may be biased to selectively maintain the first and second coupling segments in a locked configuration. The first coupling segment may include a pair of opposed openings therein, and the second coupling segment may include a pair of opposed locking tabs. The first and second coupling segments may each be tapered such that a central region of the fluid line coupling has an expanded width in relation to conduit receiving portions at opposite ends of the coupling segments, and the locking tabs may extend substantially across the expanded width.

The fluid conduit may include a first circumferential segment and a second circumferential segment. The first circumferential segment has one of translucent or opaque characteristics to prevent direct visualization of flow of exudates through the fluid conduit. The second circumferential segment has transparent characteristics to permit visualization of the flow of exudates through the fluid conduit. The first circumferential segment may have a first translucency and the second circumferential segment may have a second translucency less than the first translucency of the first circumferential segment.

The fluid conduit may define a conduit section of a predefined length with the first circumferential segment and the second circumferential segment being incorporated within the conduit section. The conduit section may include a first pair of circumferential segments and a second pair of circumferential segments. The first pair of circumferential segments has one of translucent or opaque characteristics. The second pair of circumferential segments has transparent characteristics. The circumferential segments of the first pair may be in substantial diametrical opposed relation and the circumferential segments of the second pair may be in diametrical opposed relation. In one embodiment, the second circumferential segment is dimensioned to extend through an arc segment ranging between about 60 degrees and about 120 degrees.

In another embodiment, the first circumferential segment may include an opaque segment encapsulated within the conduit wall. A plurality of opaque segments may be arranged in spaced relation within the conduit wall.

The collection canister may include at least a wall segment having one of translucent or opaque characteristics to prevent direct visualization through the wall segment of exudates collected within the collection canister and a second wall segment having transparent characteristics to permit direct visualization through the second wall segment of exudates collected within the collection canister.

A fluid portal flange may be mounted to the wound cover and adapted to couple to the fluid conduit. The fluid portal flange defines a portal opening in fluid communication with the reservoir. The fluid portal flange may include at least a flange segment having one of translucent or opaque characteristics to prevent direct visualization of exudates passing through the fluid portal flange.

The vacuum source and the collection canister may be adapted to be worn by the subject to permit the subject to be ambulatory during treatment. A control unit to control operation of the vacuum source may be mounted to the collection canister.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1A is a schematic diagram of a first embodiment of an NPWT apparatus in accordance with the present disclosure;

FIGS. 1B-1C are perspective and plan views of a vacuum port of the NPWT apparatus of FIG. 1;

FIG. 1D is a schematic diagram of a second embodiment of an NPWT apparatus in accordance with the present disclosure;

FIG. 2A is a perspective view of a first embodiment of the fluid line coupling of FIG. 1A arranged in an engaged and locked configuration;

FIG. 2B is a view similar to FIG. 2A depicting the fluid line coupling arranged in an engaged and un-locked configuration;

FIG. 2C is a partial cross sectional view of the fluid line coupling of FIG. 2A arranged in an engaged and locked configuration;

FIG. 2D is a view similar to FIG. 2C depicting the fluid line coupling arranged in an unlocked and disengaged configuration;

FIG. 3A is a perspective view of a second embodiment of a fluid line coupling arranged in an engaged and locked configuration;

FIG. 3B is a view similar to FIG. 3A depicting the fluid line coupling arranged in an un-locked and disengaged configuration;

FIG. 3C is a partial cross sectional view of the fluid line coupling of FIG. 3A arranged in an engaged and locked configuration;

FIG. 3D is a view similar to FIG. 3C depicting the fluid line coupling arranged in an unlocked and disengaged configuration;

FIG. 4A is a perspective view of a third embodiment of a fluid line coupling arranged in an engaged and locked configuration;

FIG. 4B is a view similar to FIG. 4A depicting the fluid line coupling arranged in an un-locked and disengaged configuration;

FIG. 4C is a partial cross sectional view of the fluid line coupling of FIG. 4A depicting the locking mechanism of the coupling assembly;

FIG. 4D is a partial cross sectional view of the fluid line coupling of FIG. 4A depicting the engagement mechanism of the fluid line coupling arranged in an engaged configuration;

FIG. 4E is a view similar to FIG. 4D depicting the fluid line coupling assembly arranged in a disengaged configuration;

FIG. 5A is a perspective view of a fourth embodiment of a fluid line coupling arranged in a disengaged and unlocked configuration;

FIG. 5B is an exploded perspective view of a first coupling segment of the fluid line of FIG. 5A;

FIG. 5C is a cross sectional view of the fluid line coupling of FIG. 5A;

FIG. 6A is an exploded perspective view of a fifth embodiment of a fluid line coupling;

FIG. 6B is a perspective sectional view of the fluid line coupling of FIG. 6A arranged in a locked configuration;

FIG. 7A is a perspective view of a sixth embodiment of a fluid line coupling arranged in a disengaged and unlocked configuration;

FIG. 7B is a top view of a fluid line coupling similar to the fluid line coupling of FIG. 7A arranged in an engaged and locked configuration and including a visual queue; and

FIG. 7C is top view of a fluid line coupling similar to the fluid line coupling of FIG. 7A including an alternate visual queue.

FIG. 8 is a schematic diagram of an embodiment of an NPWT apparatus including a fluid line coupling in accordance with another embodiment of the present disclosure;

FIG. 9 is a perspective view of the fluid line coupling of FIG. 8 having first and second coupling segments arranged in an engaged configuration;

FIG. 10 is a perspective view of the first coupling segment of FIG. 9 disengaged from the second coupling segment;

FIG. 11 is a perspective view of the second coupling segment of FIG. 9 disengaged from the first coupling segment;

FIG. 12 is a cross-sectional view of the fluid line coupling of FIG. 9 in the engaged configuration; and

FIG. 13 is a perspective view of the fluid line coupling of FIG. 9 from a reverse angle.

FIG. 14A is a side plan view of a section of the tubing conduit of the NPWT apparatus;

FIG. 14B is a cross-section view of the tubing conduit taken along the lines 14B-14B of FIG. 14A;

FIGS. 15-18 are cross-sectional views of an alternate embodiment of the tubing conduit; and

FIG. 19 is a view illustrating a portable pump support bag for use with the NPWT apparatus.

### DETAILED DESCRIPTION OF PREFERRED EMBODIMENTS

The wound therapy system of the present disclosure promotes healing of a wound via the use of a wound dressing, a subatmospheric pressure mechanism, and a collection canister. Generally, the subatmospheric pressure mechanism applies subatmospheric pressure to the wound to effectively remove wound fluids or exudates captured within the boundary of the composite wound dressing, and to increase blood flow to the wound bed and enhance cellular stimulation of epithelial and subcutaneous tissue. The wound therapy system may be entirely portable, i.e., it may be worn or carried by the subject such that the subject may be completely ambulatory during the therapy period. The wound therapy system including the subatmospheric pressure mechanism and components thereof may be entirely reusable or may be entirely disposable after a predetermined period of use or may be individually disposable whereby some of the components are reused for a subsequent therapy application.

The wound therapy system of the present disclosure promotes healing of a wound in conjunction with subatmospheric negative pressure therapy. The system may incorporate a variety of wound dressings, subatmospheric pressure sources and pumps, tubing's and collection canisters. The attached figures illustrate exemplary embodiments of the present disclosure and are referenced to describe the embodiments depicted therein. Hereinafter, the disclosure will be described by explaining the figures wherein like reference numerals represent like parts throughout the several views.

The wound therapy apparatus of the present disclosure promotes healing of a wound by providing a reservoir over the wound where a reduced pressure may be maintained. The reservoir subjects the wound to a negative pressure to effectively draw wound fluid, including liquid exudates, from the wound without the continuous use of a vacuum pump. Hence, negative pressure may be applied once, or in varying intervals depending on the nature and severity of the wound. A fluid line coupling of the present disclosure may facilitate disconnection of apparatus components by obstructing a fluid passageway through the coupling upon disengagement of the apparatus components. The attached figures illustrate exemplary embodiments of the present disclosure and are referenced to describe the embodiments depicted therein. Hereinafter, the disclosure will be described in detail by explaining the figures wherein like reference numerals represent like parts throughout the several views.

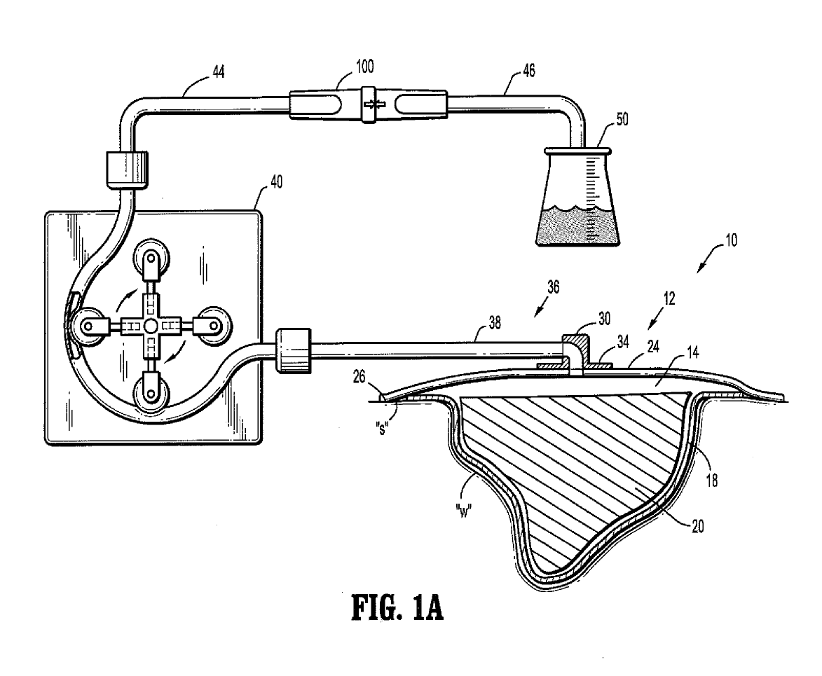

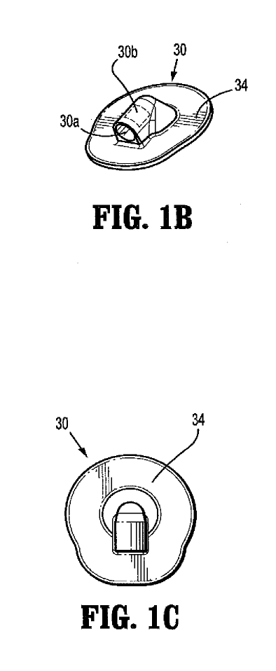

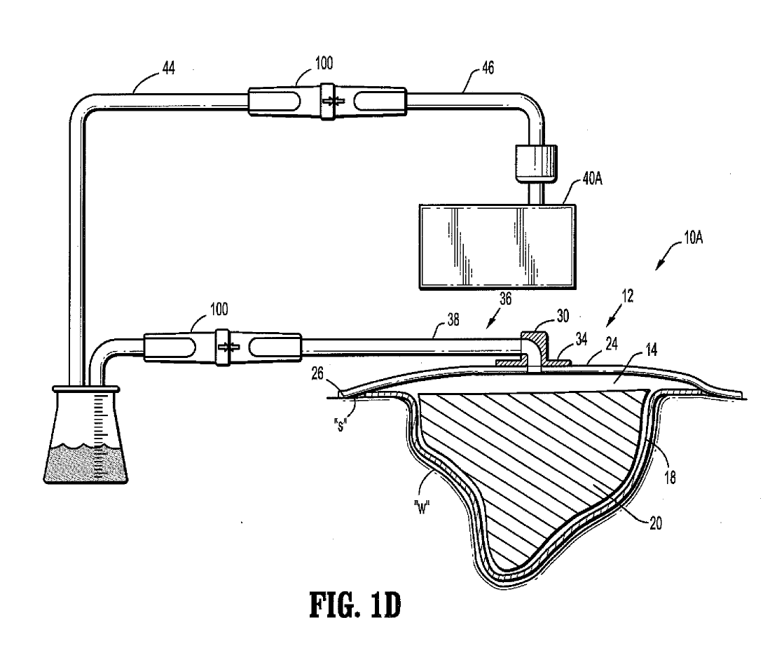

Referring initially to FIG. 1A, an NPWT apparatus according to the present disclosure is depicted generally as 10 for use on a wound “w” surrounded by healthy skin “s.” The NPWT apparatus 10 includes a wound dressing 12 positioned relative to the wound “w” to define a reservoir 14 in which a negative pressure appropriate to stimulate healing may be maintained.

Wound dressing 12 includes a contact layer 18 positioned in direct contact with the bed of wound “w” and may be formed from perforated film material. An appropriate perforated material permits the negative pressure applied to the reservoir to penetrate into the wound “w,” and also permits exudates to be drawn through the contact layer 18. Passage of wound fluid through the contact layer 18 is preferably unidirectional such that exudates do not flow back into the wound bed. Unidirectional flow may be encouraged by directional apertures formed in the contact layer 18, or a lamination of materials having absorption properties differing from those of contact layer 18. A non-adherent material may be selected such that contact layer 18 does not tend to cling to the wound “w” or surrounding tissue when it is removed. One exemplary material that may be used as a contact layer 18 is sold under the trademark XEROFLO® by Tyco Healthcare Group LP d/b/a Covidien.

Wound filler 20 is positioned in the wound “w” over the contact layer 18 and is intended to allow wound dressing 12 to absorb and capture wound fluid and exudates. Wound filler 20 is conformable to assume the shape of any wound “w” and may be packed up to the level of healthy skin “s.” The filler may be treated with agents such as polyhexamethylene biguanide (PHMB) to decrease the incidence of infection, or other medicaments to promote healing of the wound. A suitable wound filler 20 is the antimicrobial dressing sold under the trademark KERLIX™ AMD by Tyco Healthcare Group LP d/b/a Covidien.

Wound dressing 12 also includes a cover layer 24 in the form of a flexible membrane. Cover layer 24 may be positioned over the wound “w” such that a biocompatible adhesive at the periphery 26 of the cover layer 24 forms a substantially fluid-tight seal with the surrounding skin “s.” Thus, cover layer 24 may act as both a microbial barrier to prevent contaminants from entering the wound “w,” and also a fluid barrier maintaining the integrity of vacuum reservoir 14. Cover layer 24 is preferably formed from a moisture vapor permeable membrane to promote the exchange of oxygen and moisture between the wound “w” and the atmosphere. A membrane that provides a sufficient moisture vapor transmission rate (MVTR) is a transparent membrane sold under the trade name POLYSKIN®II by Tyco Healthcare Group LP d/b/a Covidien. A transparent membrane permits an assessment of wound conditions to be made without requiring removal of the cover layer 24. Alternatively, cover layer 24 may comprise an impermeable membrane 14 or a substantially rigid member.

A vacuum port 30 having a flange 34 may also be included in wound dressing 12 to facilitate connection of the wound dressing 12 to fluid conduit 36. Fluid conduit 36 defines a fluid flow path leading through the apparatus 10. The vacuum port 30 may be configured as a rigid or flexible, low-profile component, and may be adapted to receive a vacuum tube 38 in a releasable and fluid-tight manner. An adhesive on the underside of flange 34 may provide a mechanism for affixing the vacuum port 30 to the dressing 12, or alternatively flange 34 may be positioned within reservoir 14 (not shown) such that an adhesive on an upper side of the flange 34 affixes the vacuum port 30. However it is affixed to the dressing, a hollow interior of the vacuum port 30 provides fluid communication between the vacuum tube 38 and the reservoir 14. Vacuum port 30 may be provided as a pre-affixed component of dressing 16, as a component of vacuum tube 22 or entirely independently. Alternatively, vacuum port 30 may be eliminated from dressing 12 if other provisions are made for providing fluid communication with the vacuum tube 38.

One embodiment of vacuum port 30 is depicted in FIGS. 1B-1C. Vacuum port 30 may be configured as a rigid or flexible, low-profile component, and may be adapted to receive conduit 36 in a releasable and fluid tight manner. In one embodiment, vacuum port 30 includes inlet 30a which receives an end of conduit 36 in frictional relation therewith. A hollow interior of vacuum port 30 provides fluid communication between conduit 36 and the interior of wound dressing 12. Vacuum port 30 may have a valve shown schematically as reference numeral 30b built therein or in line with conduit 36, e.g., such as a one-way, zero closure or duck-bill valve to permit exudates to flow in one direction only, i.e., away from wound dressing 12. One example of a duck-bill valve will be described in detail hereinbelow. Vacuum port 30 may be provided as a pre-affixed component of wound dressing 12, as a component of conduit 36 or entirely separate and connected thereto by conventional means. Alternatively, vacuum port 30 may be eliminated if other provisions are made for providing fluid communication between wound dressing 12 and conduit 36.

Vacuum tube 38 extends from the vacuum port 30 to provide fluid communication between the reservoir 14 and vacuum source 40. Any suitable conduit may be used including those fabricated from flexible elastomeric or polymeric materials. The vacuum tube 38 may connect to the vacuum port 30, the vacuum source 40, or other apparatus components by conventional air tight means such as friction fit, bayonet coupling, or barbed connectors. The conduit connections may be made permanent, or alternatively a quick-disconnect or other releasable means may be used to provide some adjustment flexibility to the apparatus 10.

Vacuum source 40 generates or otherwise provides a negative pressure to the NPWT apparatus 10. Vacuum source 40 may comprise a peristaltic pump, a diaphragmatic pump or other mechanism that is biocompatible and draws fluids, e.g. atmospheric gasses and wound exudates, from the reservoir 14 appropriate to stimulate healing of the wound “w.” Preferably, the vacuum source 40 is adapted to produce a sub-atmospheric pressure in the reservoir 14 ranging between about 20 mmHg and about 500 mmHg, about 75 mmHg to about 125 mmHg, or about 40 mmHg. To about 80 mmHg. One suitable peristaltic pump is the KANGAROO PET™ Enteral Feeding Pump manufactured by Tyco Healthcare Group LP d/b/a Covidien.

On an exhaust side of vacuum source 40, first and second fluid conduit sections, 44 and 46 respectively, connect vacuum source 40 to a collection canister 50. First and second fluid conduit sections 44, 46 may comprise the same materials and construction as vacuum tube 38, or may assume an alternative form.

Collection canister 50 may comprise any container suitable for containing wound fluids. For example, a rigid bottle may be used as shown or alternatively a flexible polymeric pouch may be appropriate. Collection canister 50 may contain an absorbent material to consolidate or contain the wound drainage or debris. For example, super absorbent polymers (SAP), silica gel, sodium polyacrylate, potassium polyacrylamide or related compounds may be provided within canister 50. At least a portion of canister 50 may be transparent to assist in evaluating the color, quality or quantity of wound exudates. A transparent canister may thus assist in determining the remaining capacity of the canister or when the canister should be replaced.

NPWT apparatus 10 includes a fluid line coupling 100 for selectable coupling and decoupling of first and second conduit sections 44, 46. Fluid line coupling 100 may provide fluid communication between the conduit sections 44, 46 when coupled, and may obstruct fluid flow through the conduit sections 44, 46 when decoupled by sealing the conduit sections 44, 46. Thus, fluid line coupling 100 may facilitate the connection, disconnection or maintenance of components of apparatus 10, including the replacement of the collection canister 50. Alternative placement of fluid line coupling 100 at any location in line with fluid conduit 36 may facilitate other maintenance procedures. For example, placement of a fluid line coupling 100 in connection with vacuum tube 38 may facilitate servicing of vacuum source 40 without disrupting the negative pressure applied to reservoir 14.

Referring now to FIG. 1D, an alternative embodiment of the NPWT apparatus is depicted generally as 10A. Apparatus 10A demonstrates an alternate arrangement of apparatus components. In this arrangement, vacuum source 40A generates a negative pressure by drawing fluids through canister 50, which is positioned in line between the wound “w” and the vacuum source 40A. Exudates may collect in the canister while atmospheric gas continues to flow toward the vacuum source 40A. Vacuum source 40A may be configured as any of the mechanisms described with reference to vacuum source 40. Apparatus 10A also includes a plurality of fluid line couplings 100, which may be positioned in line with the fluid conduit 36 in any location in the apparatus 10A. For example, a fluid line coupling 100 may be positioned between the dressing 12 and the canister 50, between the pump 40A and the canister 50, or elsewhere depending on the configuration of the apparatus 10A.

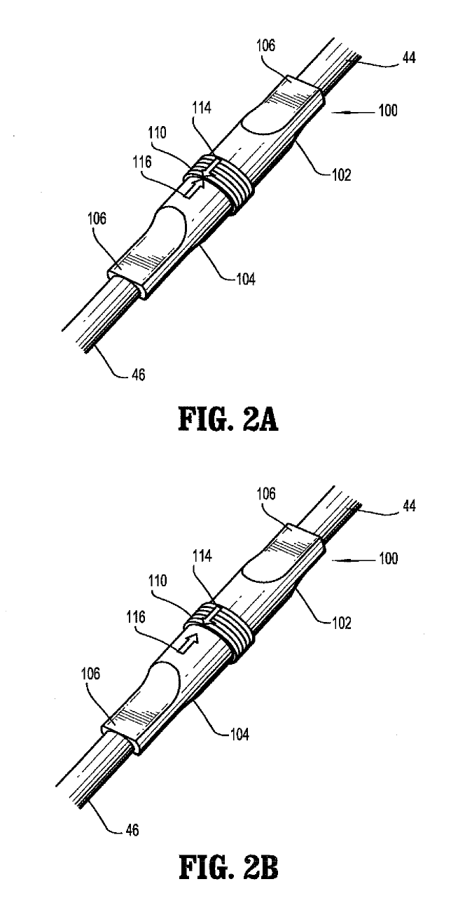

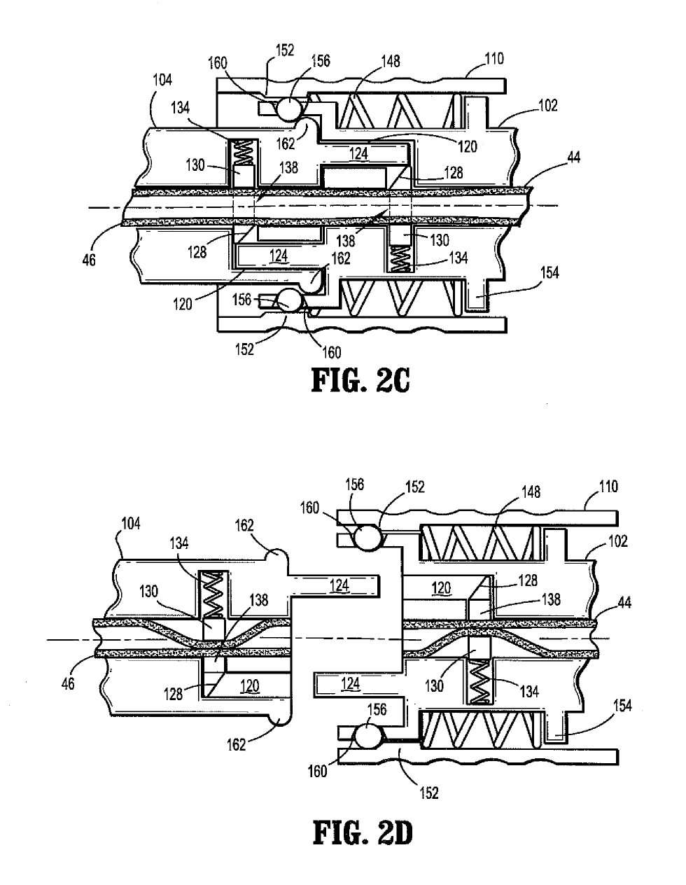

Referring now to FIG. 2A, fluid line coupling 100 may be arranged in an engaged and locked configuration to fluidly couple first and second conduit sections 44, 46. Coupling 100 includes first and second coupling segments 102, 104 each receiving a respective one of the first and second conduit sections 44, 46 therein. Coupling segments 102, 104 are each tapered at one end to provide a gripping surface 106, which may be positioned between a thumb and forefinger of an operator to facilitate engagement and disengagement of the coupling segments 102, 104. When the two coupling segments are properly engaged as depicted in FIGS. 2A and 2B, fluids may pass freely through a fluid passageway between first and second conduit sections 44, 46. First coupling segment 102 includes a locking ring 110 that may be adjusted longitudinally to move coupling 100 between a locked configuration depicted in FIG. 2A wherein first and second coupling segments 102, 104 resist separation or disengagement, and the unlocked configuration depicted in FIG. 2B wherein the two coupling segments may be separated or disengaged. A visual queue may be provided as to the engagement and locking status of the coupling 100 such as arrows 114, 116 printed, etched or otherwise applied to each of on the coupling segments 102, 104. Here, the tips of arrows 106, 108 are adjacent when first and second coupling segments 102, 104 are in the locked configuration, and substantially spaced when first and second coupling segments 102, 104 are in the unlocked configuration.

Referring now to FIG. 2C, when first and second coupling segments 102, 104 are arranged in an engaged and locked configuration, conduit portions 44, 46 are aligned to provide a fluid passageway therebetween. Preferably, a sealing engagement is established between the conduit sections 44, 46 such that fluids do not leak from the interface. When engaged, a channel 120 in each of the coupling segments 102, 104 receives a pin 124 extending from a forward face of the other of the coupling segments 102, 104. Each of the pins 124 engages a wedge shaped cam surface 128 of a post 130 positioned to protrude into channel 120. Pins 124 engage posts 130 such that the posts 130 retract against the bias of a respective biasing member such as minor springs 134. Each of the conduit portions 44, 46 is disposed through an aperture 138 in a respective post 130. The apertures 138 in each post are sized and positioned to allow the conduit portions 44, 46 to assume a natural, un-deformed shape when the coupling segments 102, 104 are engaged.

The locked configuration of fluid line coupling 100 is characterized in that locking ring 110 is moved to a forward position with respect to first coupling segment 102. A major spring 148 captured between ramps 152 on the locking ring 110 and a circumferential ledge 154 on the body of first coupling segment 102, is configured to assume a relatively relaxed condition when locking ring 110 is moved to the forward position. Thus, major spring 148 assists in moving fluid line coupling 100 to the locked configuration. Interfering members such as spherical bearings 156 are engaged by ramps 152 and are thereby caused to assume a radially inward position within tapered holes 160. Protrusions 162 extending radially outward from the second coupling segment 104 are captured by the spherical bearings 156 in the radially inward position such that first and second coupling segments 102, 104 resist longitudinal separation or disengagement. The engaged and locked configuration is the preferred condition for normal use to permit free fluid flow through the fluid passageway between fluid conduit sections 44, 46.

To facilitate removal of the collection canister 50 or otherwise disassembling NPWT apparatus 10, fluid line coupling 100 may be unlocked to permit disengagement of first and second coupling segments 102, 104. As indicated in FIG. 2D, locking ring 110 may be moved to in a direction away from second coupling segment 104 against the bias of major spring 148. Spherical bearings 156 are thus disengaged by ramps 152 and are allowed to assume a radially outward position. The radially outward position of spherical bearings 156 permits to protrusions 162 to be moved longitudinally to disengage first and second coupling segments 102, 104 from one another. As the first and second coupling segments 102, 104 are disengaged, pins 124 are withdrawn from channels 120 allowing posts 124 to assume an extended configuration assisted by the bias of minor springs 134. The apertures 138 in the posts 124 are configured such that conduit sections 44, 46 are deformed when the posts 124 are in the extended position. The deformation of the conduit sections 44, 46 obstructs fluid flow through the conduit sections 44, 46 when the two coupling segments 102, 104 are disengaged.

First and second coupling segments 102, 104 may be described as self sealing as no operator intervention is required to obstruct fluid flow through the conduit sections 44, 46 beyond the disengagement of the coupling segments 102, 104. Restricting flow through the conduit sections 44, 46 may permit the collection canister 50 to be conveniently replaced without the concern of exudates escaping through the conduit sections 44, 46. Additionally, the collection canister 50 can be replaced without disrupting the reduced pressure applied in the reservoir 14. Thus, the NPWT procedure may proceed uninterrupted while components of apparatus 10 are serviced or disconnected.

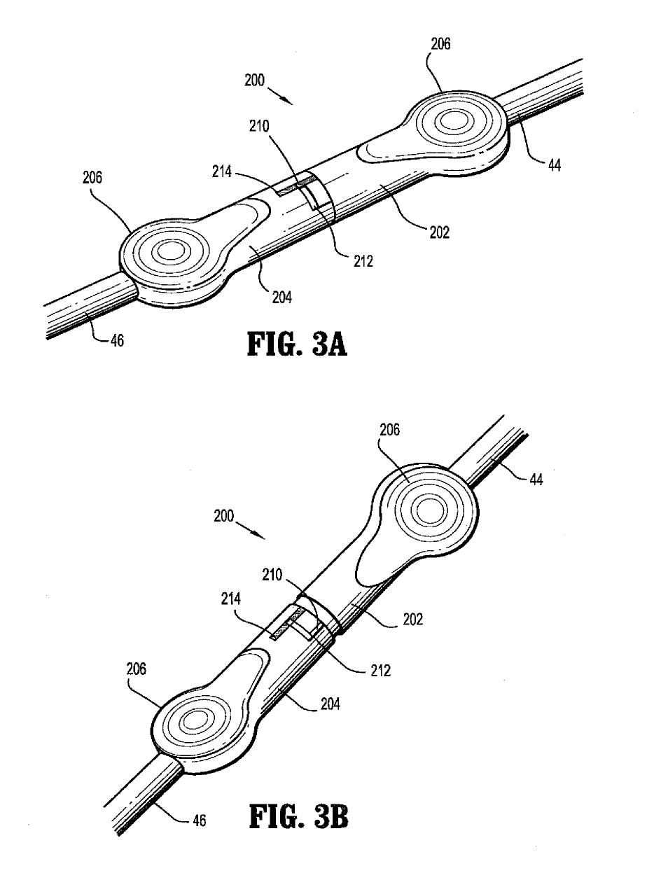

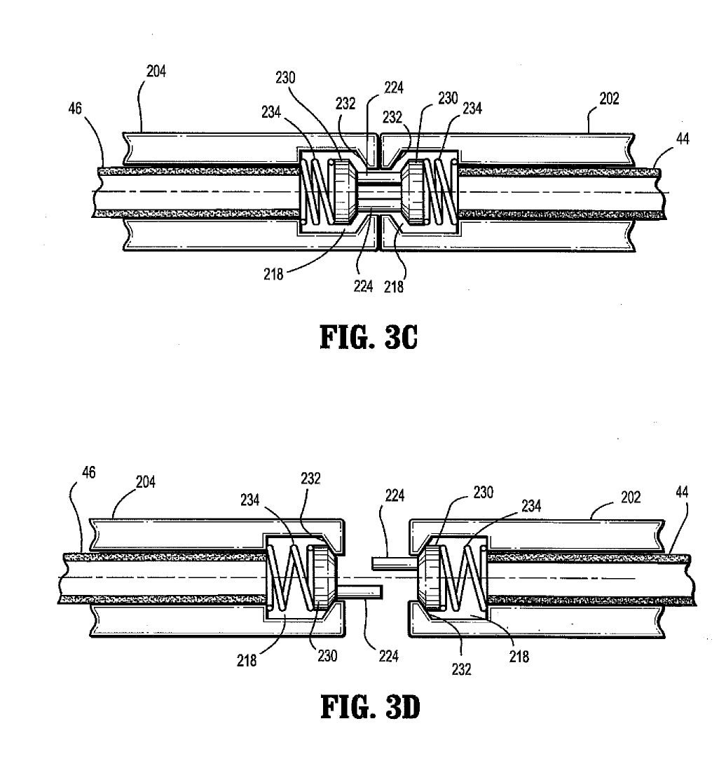

Referring now to FIG. 3A, a second embodiment of a fluid line coupling is depicted generally as 200 arranged in an engaged and locked configuration to fluidly couple first and second conduit portions 44, 46. Coupling 200 includes first and second coupling segments 202, 204 each receiving a respective one of the first and second conduit sections 44, 46 therein. Coupling segments 202, 204 are each tapered at one end to provide a gripping surface 206, which may be positioned between a thumb and a forefinger of an operator to facilitate engagement and disengagement of the coupling segments 202, 204. Gripping surfaces 206 are textured and extend laterally to assume a disk shape. This shape assists an operator in generating a torque between the coupling segments 202, 204 to allow the coupling segments 202, 204 to twist relative to one another to permit the engagement and disengagement of the coupling segments 202, 204. When the two coupling segments 202, 204 are properly engaged as depicted in FIG. 3A, fluids may pass freely between first and second conduit sections 44, 46. First coupling segment 202 includes a locking tab 210 protruding radially from an exterior surface thereof. Locking tab 210 is received in an L-shaped slot 212 in second coupling segment 204 to move coupling 200 between the engaged and locked configuration depicted in FIG. 3A, and the unlocked and disengaged configuration of FIG. 3B. This type of fastening mechanism is often referred to as a bayonet lock. A visual queue is provided by a brightly colored stripe 214 on the second coupling segment 204. A gap in the stripe indicates that coupling 200 is unlocked. The gap may be closed when brightly colored locking tab 210 moves into position and the stripe 214 is completed to indicate that the two coupling segments 202, 204 have been properly engaged and locked.

Referring now to FIG. 3C, fluid line coupling 200 is depicted in an engaged configuration permitting fluid passage between the conduit sections 44, 46. Each of the coupling segments 202, 204 includes an interior flow chamber 218 in fluid communication with a respective one of the first and second fluid conduit sections 44, 46. When the two coupling segments 202, 204 are engaged, fluid may flow freely between the two the interior flow chambers 218. Opposing pins 224 protrude from forward faces of plugs 230 disposed within flow chambers 218. Pins 224 serve to urge plugs 230 away from sealing surfaces 232 on first and second coupling segments 202, 204 against the bias of springs 234. This opens a fluid passageway between the two interior flow chambers 218 as first and second coupling segments 202, 204 are engaged. Once engaged, coupling 200 may be locked by rotating first and second coupling segments 202, 204 relative to one another to move locking tab 210 (FIG. 3A) into alignment with stripe 214. Because each pin 224 is in contact with an opposite plug 230 during this rotation, the pins 224 are adapted to slide across the forward face of the opposite plug 230. As first and second coupling segments 202, 204 are unlocked and disengaged, the bias of springs 234 urge plugs 230 against the sealing surfaces 232 to obstruct the passageway between the interior flow channels 218 as depicted in FIG. 3D. First and second coupling segments 202, 204 may thus also be described as self sealing.

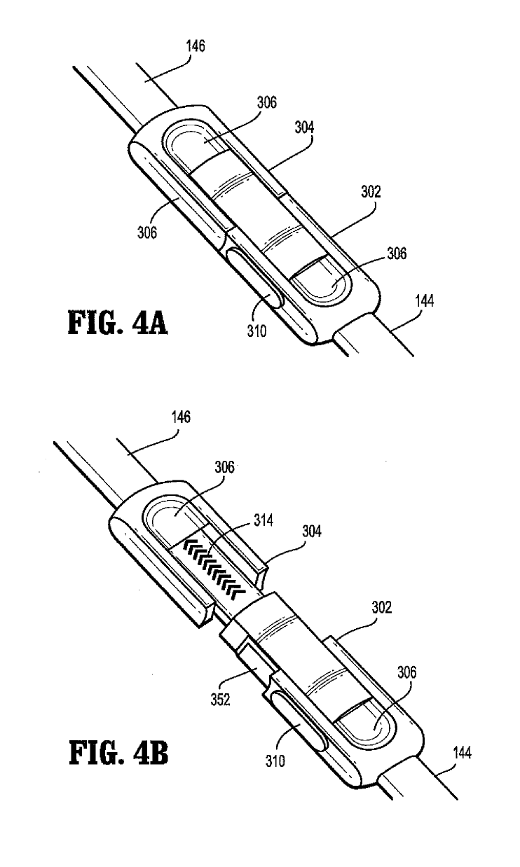

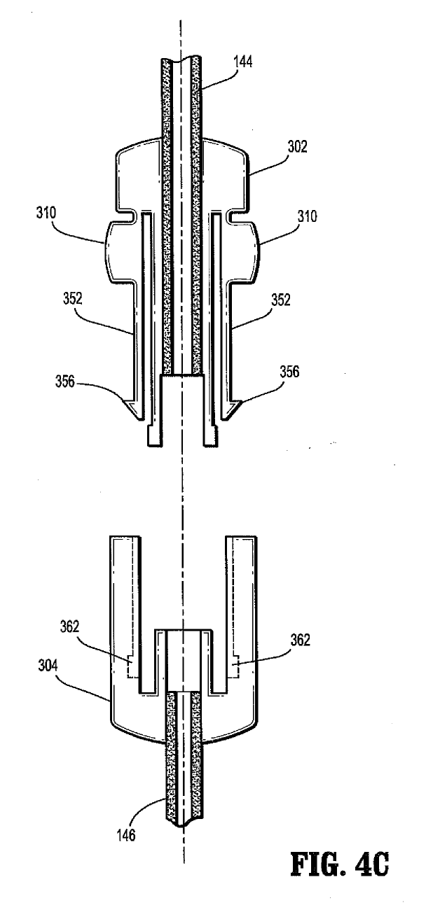

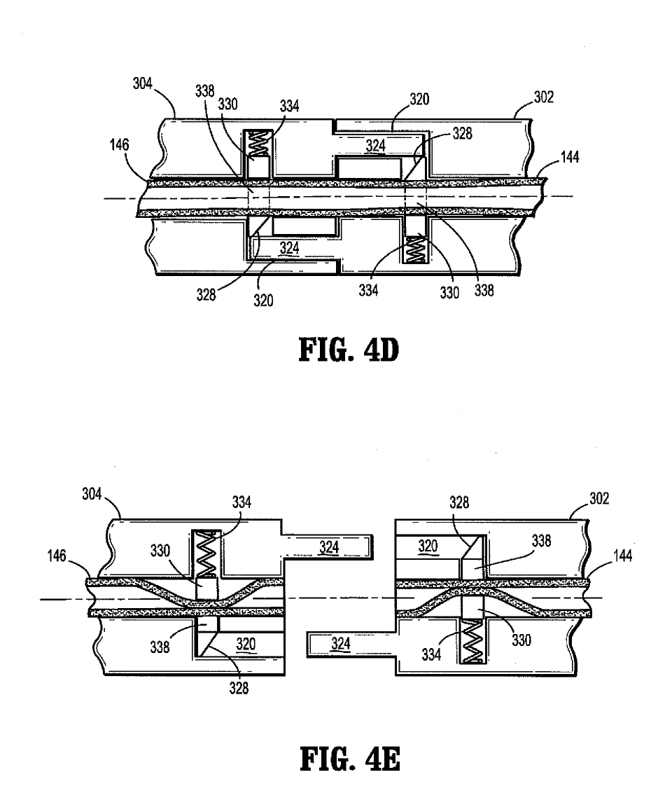

Referring now to FIG. 4A, a third embodiment of a fluid line coupling is depicted generally as 300 arranged in an engaged and locked configuration to fluidly couple first and second conduit sections 144, 146. Fluid conduit sections 144, 146 have a broader cross section than fluid conduit sections 44 and 46 to accommodate side-by-side flow channels. A multi-lumen conduit such as 144, 146 may be used in an NPWT apparatus to provide a redundancy such that fluid may flow through the conduit section 144, 146 even in the event one of the lumens becomes occluded. The occlusion of a lumen may occur, for example, as wound exudates are deposited on the interior walls of fluid conduit sections 144, 146 and accumulate until fluid flow is restricted.

Coupling 300 includes first and second coupling segments 302, 304 each receiving a respective one of the first and second conduit sections 144, 146 therein. Coupling segments 302, 304 each provide a number of depressed gripping surfaces 306 to facilitate handling by an operator to engage and disengage first and second coupling segments 302, 304. When the two coupling segments 302, 304 are properly engaged as depicted in FIG. 4A, fluids may pass freely between first and second conduit sections 144, 146. An operator may depress release button 310 to move coupling 300 into the disengaged configuration depicted in FIG. 4B. A visual queue that coupling 300 is disengaged is provided by brightly colored arrows 214 on the second coupling segment 304. These arrows are obscured by the first coupling segment 302 only when coupling 300 is properly engaged.

Referring now to FIG. 4C, the locking mechanism of coupling 300 is revealed. Release buttons 310 are coupled to elongated flexure arms 352. Each arm 352 includes a catch 356 at an end opposite the button 310 to engage a notch 362 on second coupling segment 304. When the catches 356 are engaged by the notches 362, the flexure arms are in a relatively unstressed state such that first and second coupling segments 302, 304 resist separation. When release buttons 310 are depressed, the flexure arms 352 are relatively stressed. This causes catches 356 to move inwardly, thereby releasing the catches 356 from the notches 362 providing an opportunity for separation of the first and second coupling segments 302, 304. To return coupling 300 to the engaged and locked configuration, an operator may simply approximate first and second coupling segments 302, 304. Catches 356 are equipped with a forward cam surface that cause flexure arms 352 to move to the relatively stressed state upon engagement of second coupling segment 304 without operator intervention. The stress is released as catches 356 move into notches 362. Coupling 300 may thus be described as self locking.

Referring now to FIGS. 4D and 4E, the engagement mechanism of coupling 300 is revealed. The engagement mechanism of coupling 300 may be similar to the engagement mechanism described with reference to FIGS. 2C and 2D. When first and second coupling segments 302, 304 are arranged in an engaged and locked configuration, conduit portions 144, 146 are aligned to permit fluid passage therebetween. Preferably, a sealing engagement is established between the conduit sections 144, 146 such that fluids do not leak from the interface. When engaged, a channel 320 in each of the coupling segments 302, 304 receives a pin 324 extending from a forward face of the other of the coupling segments 302, 304. Each of the pins 324 engages a wedge shaped cam surface 328 of a post 330 such that the post 330 retracts against the bias of a respective spring 334. Each of the conduit portions 144, 146 is disposed through an aperture 338 in a respective post 330. The apertures 338 in each post are sized and positioned to allow the conduit portions 144, 146 to assume a natural, un-deformed shape when the coupling segments 302, 304 are engaged.

As the first and second coupling segments 302, 304 are disengaged, pins 324 are withdrawn from channels 320 allowing posts 324 to assume an extended configuration assisted by the bias of springs 334. The apertures 338 in the posts 324 are configured such that conduit sections 144, 146 are deformed when the posts 324 are in the extended position. The deformation of the conduit sections 144, 146 may restrict flow through the conduit sections 144, 146 when the two coupling segments 302, 304 are disengaged. First and second coupling segments 302, 304 may be described as self sealing as no operator intervention is required to restrict flow through the conduit sections 144, 146 beyond the disengagement of the coupling segments 302, 304.

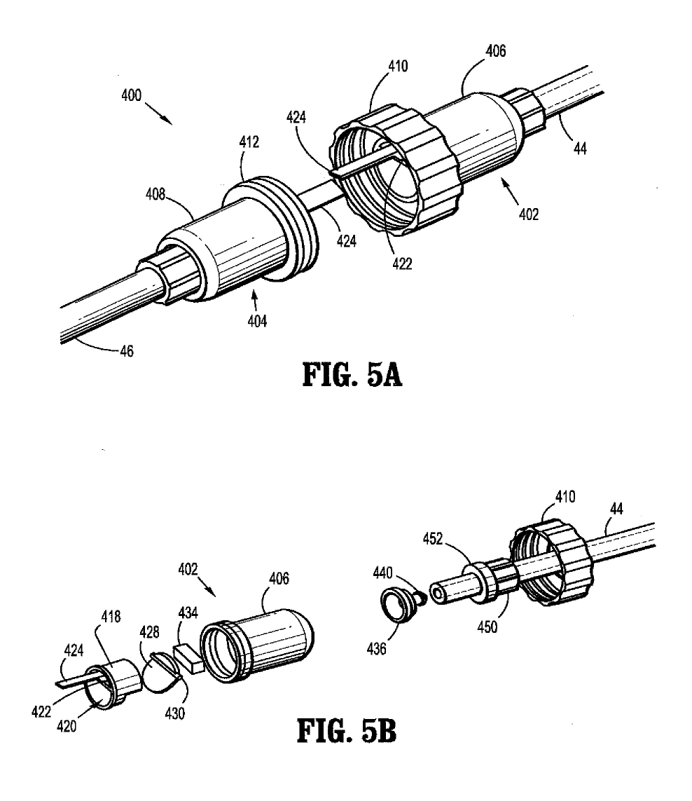

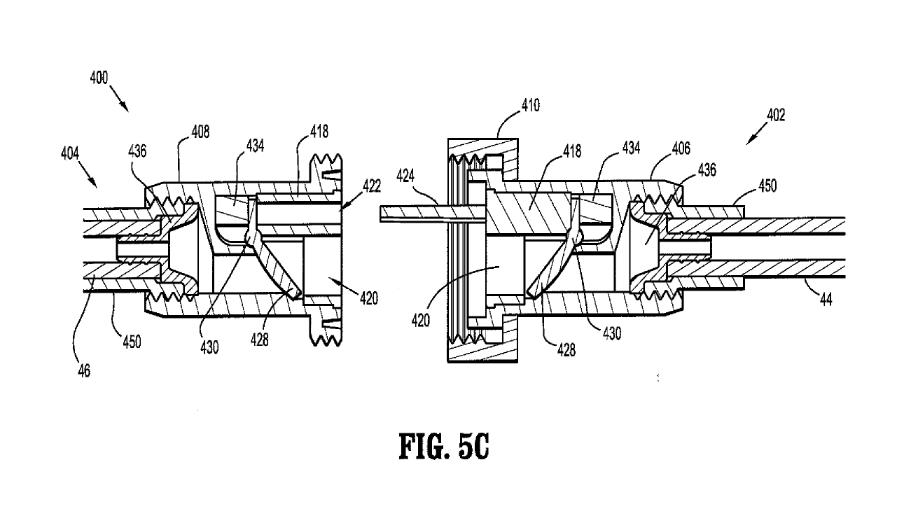

Referring now to FIG. 5A, a fourth embodiment of a fluid line coupling is depicted generally as 400 in a disengaged configuration. Coupling 400 includes first and second coupling segments 402, 404 each receiving a respective one of the first and second conduit sections 44, 46 therein. Coupling segments 402, 404 each provide a gripping component 406, 408 respectively, which may facilitate handling by an operator to engage and disengage first and second coupling segments 402, 404. When the two coupling segments 402, 404 are disengaged, fluid flow is restricted through first and second conduit sections 44, 46, but when coupling segments 402, 404 are properly engaged, fluids may pass freely between first and second conduit sections 44, 46. A locking nut 410 provides a locking mechanism for fluid line coupling 400. Locking nut 410 is positioned loosely around gripping component 406 of first coupling segment 402 such that it may rotate freely thereabout. Exterior threads 412 on gripping component 408 of second coupling segment 404 may be engaged by locking nut 410 to maintain coupling segments 402, 404 in a locked and engaged configuration.

Referring now to FIG. 5B, interior components of the gripping section 406 of first coupling segment 402 are revealed. A forward component 418 defines a flow channel 420 therein which may carry wound fluids and atmospheric gasses when first and second coupling segments 402, 404 are engaged. A pin receiving channel 422 extends through the forward component 418, and a pin 424 protrudes from a forward face of the forward component 418. Pin receiving channel 422 is adapted to receive a pin 424 (FIG. 5A) from a similar forward component 418 provided with second coupling segment 404.

A flipper door 428 is positioned adjacent the forward component 418. Flipper door 428 may be substantially rigid component with an angled geometry such that flipper door 428 may rotate about pivot 430. A rubber spring 434 may be provided between flipper door 428 and an interior surface of gripping component 406 to bias flipper door 428 to a closed position. Rubber spring 434 may be formed from a solid mass of foam rubber or another resilient or elastic material that, when compressed, has a tendency to return to an undeformed state.

A nozzle 436 is coupled to an open end of first fluid conduit section 44. Nozzle 436 is equipped with annular barbs 440 protruding from an exterior surface thereof such that barbs 440 may be positioned within conduit 44 to deform an interior surface of the conduit section 44 and form a fluid tight interface therewith. A collar 450 may be positioned around an exterior surface conduit 44. Exterior threads 452 on collar 450 may interface with corresponding threads within gripping section 406 to secure first coupling segment 402 to first conduit section 44.

Referring now to FIG. 5C, second coupling segment 404 is arranged to compliment first coupling segment 402. Gripping component 408 of second coupling segment 404 houses a forward component 418, flipper door 428, rubber spring 434, nozzle 436 and collar 450 in a manner similar to gripping component 406 of first coupling segment 402 as described above.

When first and second coupling segments 402 and 404 are disengaged, rubber springs 434 bias flipper doors 428 such that flipper doors 428 seat against interior surfaces of respective gripping components 406, 408. The flow of fluids through flow channels 420 is thereby restricted. Upon engagement of first and second coupling segments 402 and 404 however, flipper doors 428 may rotate about pivots 430 to open flow channels 420. The pins 424 are received in opposite pin receiving channels 422 and extend through forward components 418. The pins 424 contact flipper doors 428 and cause the flipper doors 428 rotate about pivots 430 against the bias of the rubber springs 434. Fluid communication is thereby provided between first and second fluid conduit sections 44, 46 through fluid line coupling 400 when first and second coupling segments 402 and 404 are engaged.

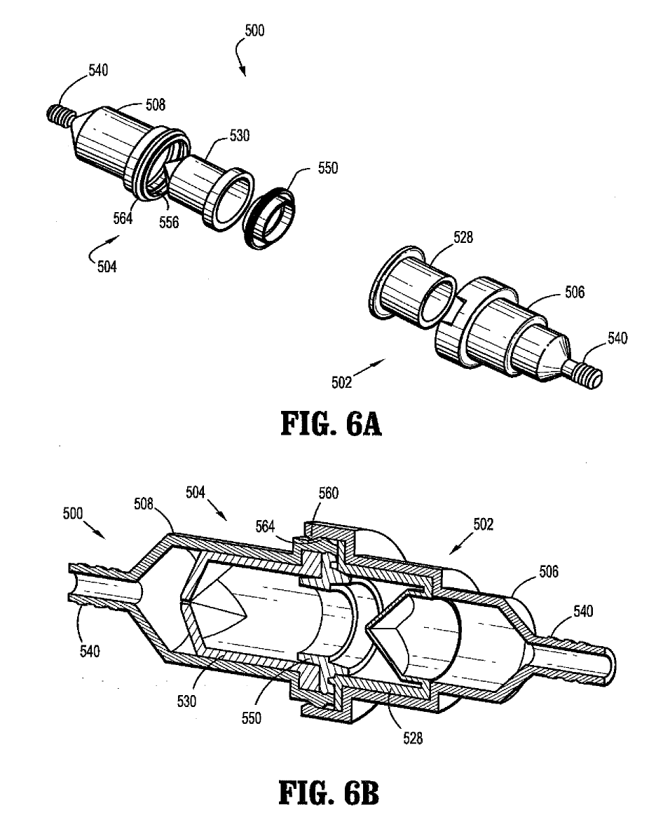

Referring now to FIG. 6A, a fifth embodiment of a fluid line coupling is depicted generally as 500. Fluid line coupling 500 includes first and second coupling segments 502, 504 respectively. First coupling segment 502 includes a gripping component 506 and second coupling segment 504 includes a gripping component 508. Gripping components 506, 508 may be handled by an operator to engage and disengage fluid line coupling 500.

A first duckbill valve 528 is housed within gripping component 506 and a second duckbill valve 530 is housed within second gripping component 508. A duckbill valve such as 528 and 530 typically includes a pair of lips arranged in a converging relationship formed from a resilient material. The lips are generally biased to abut one another so as to form a slit therebetween. When placed in a fluid flow path, the flow pressure against the resilient lips in a first direction opens the slits allowing fluid to pass freely therethrough. Flow pressure in an opposite direction, however, tends to close the slit preventing fluid flow in the opposite direction.

Gripping components 506 and 508 are each equipped with annular barbs 540. Annular barbs 540 permit attachment to fluid conduit sections in a manner similar to annular barbs 440 described with reference to FIG. 5B above. Gripping component 508 may receive a retainer 550 to secure second duckbill valve 530 therein. An annular rim 554 is provided on an exterior surface of retainer 550. An annular notch 556 on an interior surface of gripping portion 508 receives annular rim 554 such that retainer 550 may be secured within gripping portion 508 to maintain the position of second duckbill valve 530 therein.

Referring now to FIG. 6B, a locking mechanism may be provided by a pair of complementary annular tabs 560, 564. Tab 560 on gripping component 506 engages tab 564 on gripping component 508 in a snap-fit manner to maintain first and second coupling segments 502 504 in an engaged configuration. When engaged, fluid may pass freely in a direction from first coupling segment 502 to second coupling segment 504 due to flow pressure in that direction, which may serve to open first and second duckbill valves 528, 530. For example, if fluid line coupling 500 is arranged in an engaged configuration on a fluid conduit between a wound dressing and a vacuum source, a reduced pressure supplied by the vacuum source downstream of the coupling 500 may serve to open the duckbill valves 528, 530.

When first and second coupling segments 502, 504 are disengaged, fluid flow may be interrupted. The resiliency of first and second duckbill valves 528, 530 may be sufficient to close the valves 528, 530 when first and second coupling segments 502, 504 are disengaged. Atmospheric pressure downstream of first duckbill valve 528 may also assist in closing first duckbill valve 528 since the atmospheric pressure may be greater than the reduced pressure applied upstream of first duckbill valve 528. Thus, first and second duckbill valves may provide an engagement mechanism by permitting fluid to flow through fluid line coupling 500 when first and second coupling segments 502, 504 are engaged, and interrupting fluid flow when first and second coupling segments 502, 504 are disengaged.

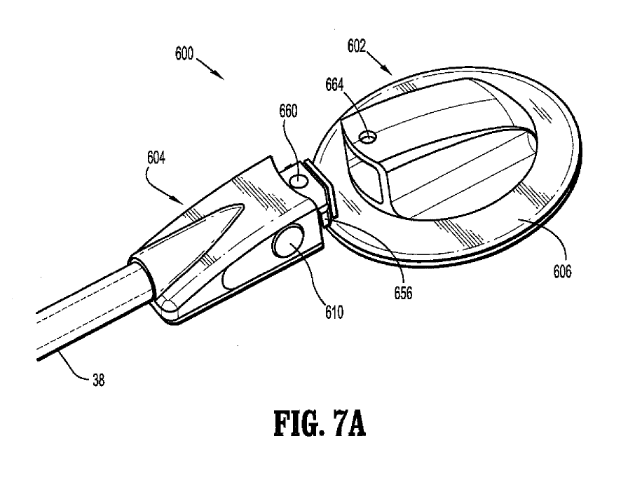

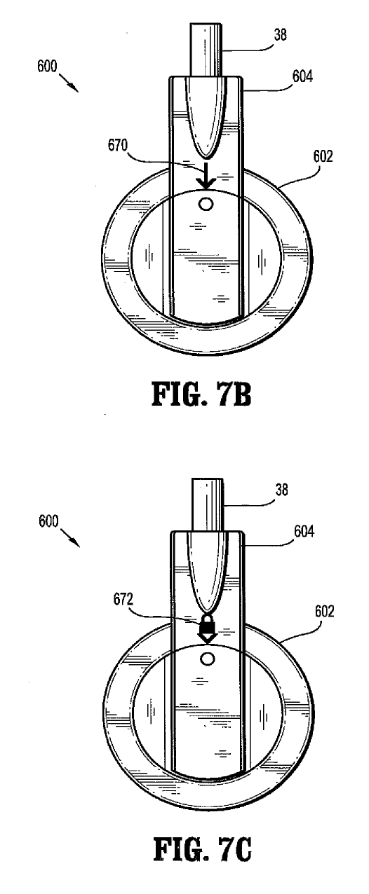

Referring now to FIG. 7A, a sixth embodiment of a fluid line coupling is depicted generally as 600. Unlike the fluid line couplings 100, 200, 300, 400 and 500 described above that are adapted to couple a first conduit section, e.g., 44 to a second fluid conduit section, e.g., 46, fluid line coupling 600 may serve to couple reservoir 14 to a fluid conduit such as vacuum tube 38 described above with reference to FIG. 1A. Fluid line coupling 600 includes a first coupling segment 602, which assumes the form of a vacuum port similar to vacuum port 30, also described above with reference to FIG. 1A. A second coupling segment 604 is adapted to receive vacuum tube 38 therein and form a fluid tight interface therewith.

First coupling segment 602 includes an elastomeric flange or skirt 606 that may be affixed to a wound dressing, such as wound dressing 12, by an adhesive film overlay, such as cover layer 24. In this manner, first coupling segment 602 may communicate with reservoir 14.

Second coupling segment 604 may provide a locking mechanism similar to the locking mechanism described with reference to FIG. 4C above. A pair of push buttons 610 may be operatively associated with a pair of catches 656. Catches 656 may engage notches (not shown) on an interior surface of first coupling segment 602 in an unstressed state to maintain first and second coupling segments 602, 604 in an engaged configuration. Push buttons 610 may then be depressed to release catches 656 from the notches to permit first and second coupling segments 602, 604 to be disengaged.

Any of the engagement mechanisms described above may be incorporated into fluid line coupling 600 to interrupt fluid flow when first and second coupling segments 602, 604 are disengaged. For example, an engagement mechanism such as a duckbill valve as described above with reference to FIG. 6A may be incorporated into first coupling segment 602 such that fluids may not flow through first coupling segment 602 when first and second coupling segments 602, 604 are disengaged. Second coupling segment 604 may incorporate an engagement mechanism such as that described with reference to FIGS. 2C and 2D in which vacuum tube 38 may be deformed upon disengagement of first and second coupling segments 602, 604. Alternatively, fluid line coupling 600 may permit fluid flow through one or both of first and second coupling segments 602, 604.

A visual queue may be provided as to the engagement status of first and second coupling segments 602, 604 such as a green indicator 660 that is fully visible through a window 664 only when first and second coupling segments 602, 604 are properly engaged. Other visual queues may be provided on fluid line coupling 600 as depicted in FIGS. 7B and 7C. An arrow may be applied to second coupling segment 604 as depicted in FIG. 7B to indicate that the second coupling segment 604 should be inserted into the first coupling segment 602. Alternatively, a figure of a padlock may be applied to second coupling segment 604 as depicted in FIG. 7C to indicate that first and second coupling segments 602, 604 may be locked in an engaged configuration.

Any of the engagement and locking mechanisms described herein may be incorporated into fluid line couplings as described above, or any of these mechanisms may be used interchangeably to form a fluid line coupling suitable for a particular application. The fluid line couplings may serve to couple two distinct conduit sections, e.g. 44 and 46 disposed between a vacuum source 40 and canister 50, or in another location such as between a wound dressing 12 and a canister. It is also contemplated that the engagement and locking mechanisms may be incorporated into other apparatus components such as, for example, an inlet to a vacuum source such as vacuum source 40.

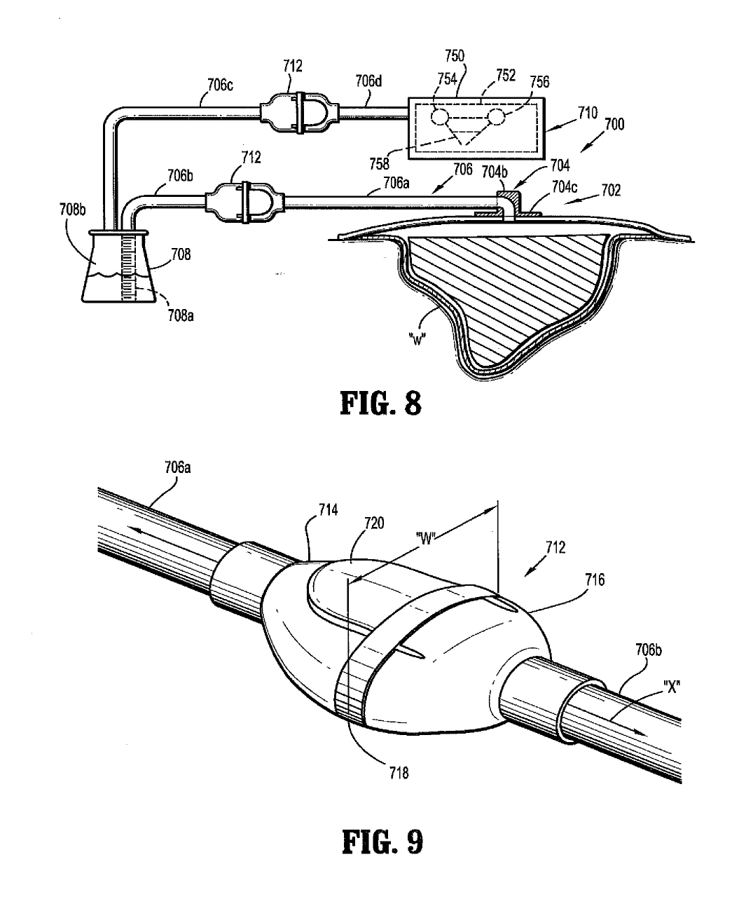

Referring initially to FIG. 8, an NPWT apparatus in accordance with another embodiment of the present disclosure is depicted generally as 700 for use on a wound “w” surrounded by healthy skin “s.” The NPWT apparatus 700 includes a wound dressing 702 which may be substantially similar to the wound dressing of the embodiment of FIG. 1.

Vacuum port 704 may be adapted to receive a fluid conduit 706 in a releasable and fluid-tight manner to provide fluid communication between the fluid conduit 706 and the reservoir beneath wound dressing 702. Fluid conduit 706 defines a flow path though the apparatus 700 for fluids such as wound exudates and atmospheric gasses. Vacuum port 704 may be eliminated from dressing 702 if other provisions are made for providing fluid communication with the fluid conduit 706.

The fluid conduit 706 includes several distinct segments 706A, 706B, 706C and 706D. A first fluid conduit section 706 extends from the vacuum port 704, and the other segments 706B, 706C and 706D extend to and from other components of the apparatus 700. Any suitable conduit may be used including those fabricated from flexible elastomeric or polymeric materials. The fluid conduit sections may connect to the vacuum port 704 and the other components of the apparatus 700 by conventional air tight means such as friction fit, bayonet coupling, or barbed connectors. The connections may be made permanent, or alternatively a quick-disconnect or other releasable means may be used to provide some adjustment flexibility to the apparatus 10.

Fluid conduit section 706B leads into a collection canister 708. Collection canister 708 may comprise any container suitable for containing wound fluids. For example, a rigid bottle may be used as shown or alternatively a flexible polymeric pouch may be appropriate. Collection canister 708 may contain an absorbent material to consolidate or contain the wound drainage or debris. For example, super absorbent polymers (SAP), silica gel, sodium polyacrylate, potassium polyacrylamide or related compounds may be provided within canister 708. At least a portion of canister 708 may be transparent to assist in evaluating the color, quality or quantity of wound exudates. A transparent canister may thus assist in determining the remaining capacity of the canister or when the canister should be replaced.

On an exhaust side of the collection canister 708, fluid conduit sections 706C and 706D connect collection canister 708 to a subatmospheric pressure mechanism 710 that generates or otherwise provides a negative pressure to the NPWT apparatus 700.

NPWT apparatus 700 includes a fluid line coupling 712 for selectable coupling and decoupling of conduit sections 36A and 36B. Fluid line coupling 712 may maintain fluid communication between the conduit sections 706A, 706B when engaged, and may interrupt fluid flow between the conduit sections 706A, 706B when disengaged. Thus fluid line coupling 712 may facilitate the connection, disconnection or maintenance of components of apparatus 700, including the replacement of the collection canister 708. Additional or alternative placement of fluid line coupling 712 at any location in line with fluid conduit 706 may facilitate other maintenance procedures. For example, placement of a fluid line coupling 712 between fluid conduit sections 706C and 706D, as depicted, may facilitate servicing of vacuum source 710.

Referring now to FIG. 9, fluid line coupling 712 may be arranged in an engaged and locked configuration to fluidly couple first and second conduit sections 706A, 706B. A fluid passageway through the coupling 712 defines a longitudinal axis “X.” Coupling 712 includes first and second coupling segments 714, 716 each receiving a respective one of the first and second conduit sections 706A, 706B therein in a fluid tight manner. Fluid conduit sections 706A and 706B may be permanently affixed to their respective coupling segments 714, 716 for example, with an appropriate adhesive, or may be releasably engaged therewith. When the two coupling segments 706A, 706B are properly engaged, fluids may pass freely through a fluid passageway between first and second conduit sections 706A, 706B.

Coupling segments 714, 716 are each tapered such that a central region of the coupling 712 has an expanded width “W” in relation to the opposite ends wherein the respective fluid conduit sections 706A, 706B are received. In this way, a gripping surface 718 is provided which may be conveniently positioned between a thumb and forefinger of an operator to handle the coupling 712. A pair of broad locking tabs 720 are also provided, which extend substantially across the expanded width “W” of the coupling 712. Locking tabs 720 are cantilevered from second coupling segment 716 such that the locking tabs 720 may be depressed toward one another to facilitate the connection and disconnection of the two coupling segments 714, 716.

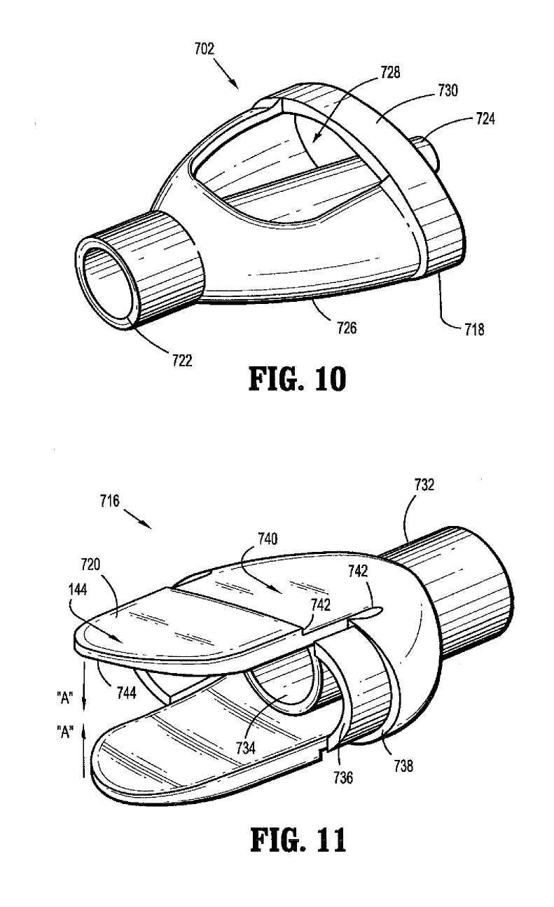

As depicted in FIG. 10, first coupling segment 714 includes a hollow conduit receiving portion 722 at one end of the coupling segment 714, which is in fluid communication with a tapered projection 724 extending longitudinally to an opposite end of the coupling segment 714. The tapered projection 724 extends through a central region of the coupling segment 714, and is encircled by an outer wall 726 extending both radially and longitudinally from the conduit receiving portion 722. The outer wall 726 includes a pair of openings 728 therein positioned on opposite lateral ends of the first coupling segment 714. The openings 728 approximate the size and shape of the locking tabs 720 of the coupling segment 716. Extending longitudinally beyond the openings 728 to a forward end of the outer wall is locking rim 730. The locking rim 730 may partially form gripping surface 718.

As depicted in FIG. 11, the second coupling segment 716 includes a hollow conduit receiving portion 732, which is in fluid communication with a cylindrical channel 734. The cylindrical channel 734 extends through a central region of the coupling segment 716, and is encircled by an inner wall 736 and an outer wall 738 extending both radially and longitudinally from the conduit receiving portion 722. The inner wall 736 extends between the two locking tabs 720 on opposite lateral sides of the coupling segment 716, and extends longitudinally beyond the outer wall 738.

The locking tabs 720 protrude from the outer wall 738 at a cantilevered support end 140, and each include a locking ledge 742 extending transversely across the coupling segment 716. Slots 742 project into the outer wall 738 promoting flexibility of the locking tabs 720. The locking tabs 720 terminate at a free end 744 opposite the cantilevered support end 740, where control surfaces 746 are located. The control surfaces 746 provide a finger-contact area that an operator may engage to flex the locking tabs 720 in the direction of arrows “A” to move the locking ledges 742 from a first position to a second position. The locking ledges 742 are readily movable as they located between the control surfaces 746 and the cantilevered support end 140.

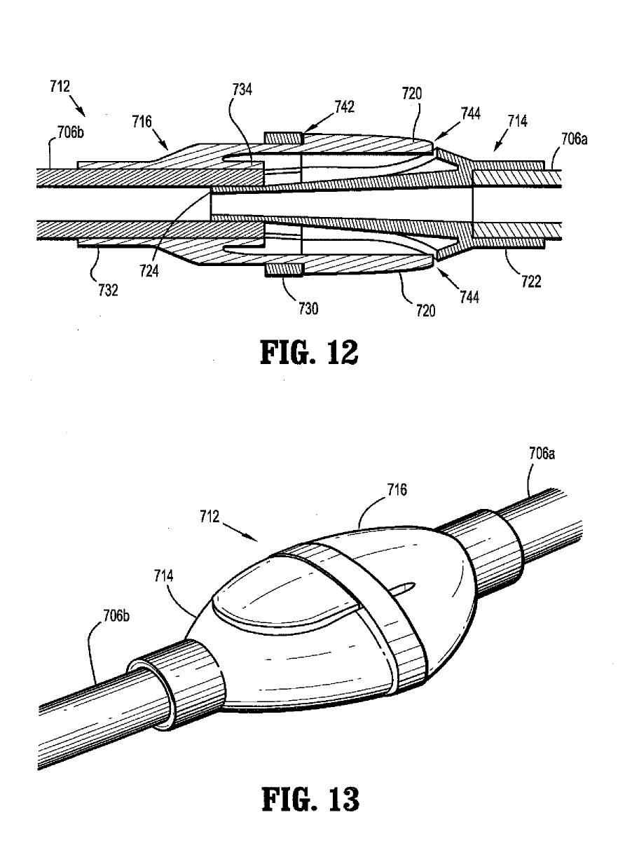

When first and second coupling segments 714, 716 are engaged as depicted in FIGS. 12 and 13, fluid communication is established between fluid conduit sections 706A and 706B through the coupling 712. The circular channel 734 receives the tapered protrusion 724 in a fluid-tight manner through a friction fit or a similar connection. The control surfaces 746 of the second coupling segment 716 protrude through the openings 728 on the first coupling segment 714. The locking tabs 720 are spring biased such that the locking ledges 742 are in the first position where they may be engaged by the locking rim 730 to maintain the connection between the first and second coupling segments 714, 716.

To facilitate removal of the collection canister 708 or otherwise disassembling NPWT apparatus 10, fluid line coupling 712 may be unlocked to permit disengagement of first and second coupling segments 714, 716. To separate first and second coupling segments 714, 716 an operator may grip the fluid line coupling 712 about gripping surfaces 718 while flexing locking tabs 720 radially inwardly. Since the locking tabs 720 are broad to extend substantially across the expanded width “W” of the coupling 712, the operator may conveniently press a finger or thumb through the opening 728 in the first coupling segment 714 until the locking ledges 742 on the locking tabs 720 have moved to the second position radially inward of the locking rim 730. Since the free ends 744 of the cantilevered locking tabs 720 are spaced furthest from slots 742, a generous moment assists the user in moving the locking ledges 742 to the second position. Once the locking ledges 742 are in the second position, the first and second coupling segments may be separated longitudinally to interrupt the fluid flow between the first and second conduit sections 706A and 706B.

To reestablish the fluid passageway between the first and second conduit sections 706A and 706B, the fluid line coupling may be reassembled with a single longitudinal motion. The free ends 744 of the locking tabs 720 may be placed within the locking rim 730, and thereafter the curvatures of the first and second coupling segments 714, 716 may guide the coupling segments 714, 716 into proper alignment as the coupling segments 714, 716 are longitudinally approximated. The locking rim 730 will cause the locking tabs to flex radially inwardly until the locking ledges 742 move into the openings 728. The bias of the locking tabs 720 will then cause the locking ledges 742 to return to the first position in which the fluid line coupling 712 is engaged and locked. In this configuration, the inner wall 736 will extend into the locking rim 730.

The engagement and locking mechanisms described herein serve to couple two distinct conduit sections, e.g. 706A and 706B disposed between a subatmospheric pressure mechanism or vacuum source 710 and canister 708. It is also contemplated that the engagement and locking mechanisms may incorporated into other apparatus components such as, for example, vacuum port 704.

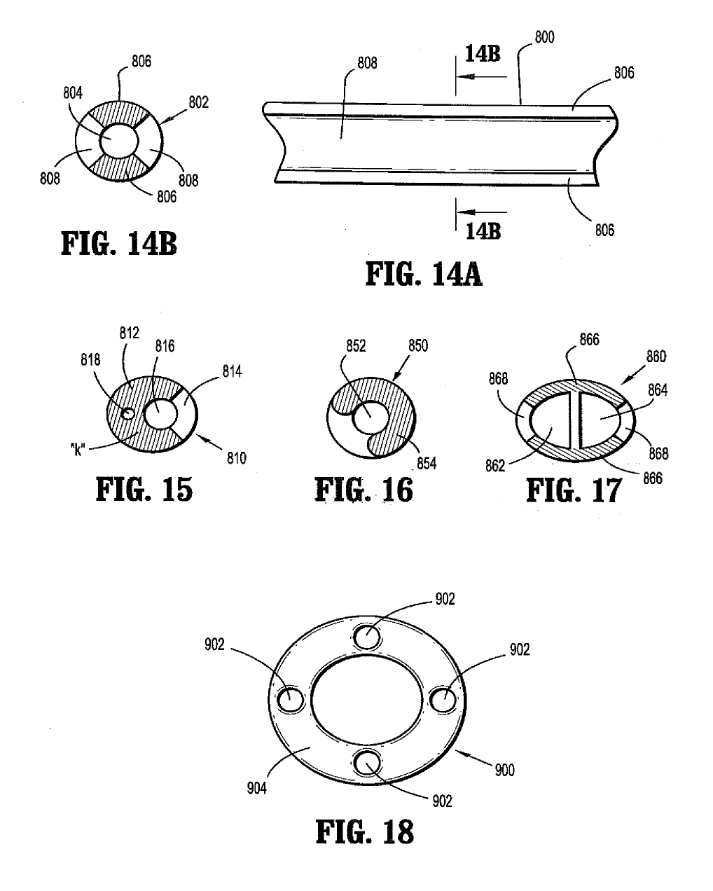

Referring now to FIGS. 14A-14B, one embodiment of conduit 800 will be discussed. Conduit 800 may be a flexible tubing made from a suitable polymeric material through, e.g., known extrusion processes. Suitable materials for tubing include polyurethane, polyethylene, polystyrene, flexible polyvinyl chloride (PVC) or the like. Conduit 800 has outer circumferential or peripheral wall 802 defining central lumen 804 which receives and transfers the flow of exudates from wound dressing 102 to collection canister 806. Outer wall 802 may define a number of circumferential wall or arc segments having various transparent, translucent or opaque characteristics which selectively permit, limit or prevent viewing of the fluids or exudates passing through lumen 804. For example, outer wall 802 may include a first pair of opposed arc segments 806 having a first visual characteristic and a second pair of opposed arc segments 808 having a second visual characteristic different from the first visual characteristic. In one embodiment, first pair of arc segments 806 has one of a translucent, semi-opaque or opaque characteristic substantially limiting or impeding visualization through the arc segments 806. Second pair of arc segments 808 may be substantially clear or slightly translucent thereby permitting visualization through these arc segments 808 for inspection of the tube contents. By virtue of this arrangement, the visual impact, including induced stress on the subject, associated with viewing discolored wound exudate through an entirely clear conduit 800 is minimized; however, visual detection of fluids within conduit 800 is permitted, although on a limited basis, through second pair of arc segments 808. This enables the subject to discretely visually detect the presence of fluids or clogs within conduit 800 to ensure proper operation and drainage of the wound exudate.

In one embodiment, the translucent, semi-opaque or opaque characteristics of the first pair of arc segments 806 of conduit 800 may be provided through a tube frosting process or technology on the tube surface. The frosting technology may be applied to selectively frost first pair of arc segments 800 of conduit 800. A frosted surface finish is effected by introducing turbulent air flow across desired arc segments, e.g., arc segments 806, of the conduit 800 during, or subsequent to, the extrusion process. The second pair of arc segments 808 not subjected to the turbulent air flow will remain non frosted or relatively clear.

In the embodiment of FIGS. 14A-14B, each arc segment 806, 808 extends through about a 90 degree arc; however, it is also envisioned that other arrangements are also envisioned. For example, FIG. 15 illustrates conduit 810 having a single translucent or opaque arc segment 812 ranging through an arc of about 240 degrees to about 270 degrees. Conduit arc segment 814 is substantially clear or slightly translucent and correspondingly ranges through an arc segment of about 90 degrees to about 120 degrees. This arrangement is less discrete than the prior embodiment and defines a single stripe for permitting viewing into lumen 816 and of the exudates. In further accordance with this embodiment, lumen 816 is radially offset relative to the central longitudinal axis “k”. In addition, a second lumen 818 extends within conduit 810 to permit additional functions such as pressure sensing, irrigation, controlled wound ventilation or the like.

FIG. 16 illustrates an embodiment of conduit 850 where lumen 852 is centrally located and where the translucent or opaque arc segment 854 extends through an arc segment of about 240 degrees to about 270 degrees. The translucent segment could be a surface treatment rather than a full thickness wall section.

FIG. 17 illustrates a conduit having a substantially elliptical or oval cross-section. Conduit 860 defines first and second lumens 862, 864. Each lumen 862, 864 may be intended for drainage of wound exudates thereby providing a level of redundancy to the system in the event of clogging of either of the lumens 862, 864. In the alternative, one lumen may be in fluid communication with an irrigant source for supply medicinal or the like to the wound. Outer wall of conduit includes first and second pairs of diametrically opposed arc segments 866, 868. First arc segments 866 are translucent or opaque while second arc segments are generally clear 868.

FIG. 18 illustrates an alternate embodiment where conduit 900 is substantially transparent, but, includes at least one or more opaque segments 902 at least partially embedded, or fully contained, within wall 904 of the conduit 900. Opaque segments 202 may be round, elliptical, rectangular or any other geometrical shape in cross-section. Opaque segments 902 may extend along at least a portion of the length of the tubing 900 or along the full length, i.e., the opaque segments may be rod like. Opaque segments 902 may be radially spaced about the longitudinal axis “k” of conduit 900 whereby adjacent opaque segments are spaced a predetermined distance. This spacing permits at least partial visualization within the interior of conduit 900. Opaque segments 902 may be arranged in equal intervals about the longitudinal axis “k”. Other arrangements are also envisioned.

With reference again to FIG. 8, it is also envisioned that vacuum port 704, fluid line coupling 712, and/or collection canister 708 may be translucent or opaque in whole or in part to permit limited and/or selective viewing through these components. For example, vacuum port 704 may have one section 704B which may be transparent and a second section 704C (the flange) being relatively translucent or semi-opaque. Collection canister 708 may also include transparent section 708a adjacent the gradation markings to permit viewing of the level of the exudates and translucent section 706B covering the remainder of the collection canister 706 (FIG. 8).

The translucent, semi-opaque or opaque characteristics may be effected through other methodologies in lieu of frosting techniques. For example, selective arc segments of the tubing conduit may be colored by adding a dye or color during extrusion to provide the desired level of translucency or opaqueness. Other means are also envisioned.

Referring again to FIG. 8, the remaining component of wound therapy system 700 will be discussed. Subatmospheric pressure mechanism 710 may include a housing 750 and control unit 752 (shown in schematic) disposed within the housing 750. Housing 750 may be any suitable rigid or flexible member. Housing 750 may be adapted for donning by the subject. Control unit 752 may incorporate vacuum source 754, power source 756 and logic or circuitry 758 for controlling operation of the unit 252. Vacuum source or pump 754 generates or otherwise provides negative pressure to wound therapy system 700. Vacuum source or pump 174 may be a pump of the diaphragmatic, peristaltic or bellows type or the like, in which the moving part(s) draw exudates out of the wound bed “w” into the wound dressing 102 by creating areas or zones of decreased pressure e.g., vacuum zones with the wound dressing 102. This area of decreased pressure preferably communicates with the wound bed “w” to facilitate removal of the fluids.

Vacuum source or pump 754 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg and about 125 mmHg, or between about 30 mmHg and 80 mmHg. Vacuum source or pump 754 is actuated by an actuator which may be any means known by those skilled in the art, including, for example, AC motors, DC motors, voice coil actuators, solenoids, and the like. The actuator may be incorporated within pump 754.

Power source 756 may be disposed within housing 750 or separately mountable to the housing 750. A suitable power source 756 includes alkaline batteries, wet cell batteries, dry cell batteries, nickel cadmium batteries, solar generated means, lithium batteries, NiMH batteries (nickel metal hydride) each of which may be of the disposable or rechargeable variety.

Collection canister 708 collects exudates “e” removed from the wound bed “w” during therapy through conduit or tubing 706. Collection canister 708 may be associated with housing 750 and may be incorporated within the housing 750 or releasably connected to the housing 750 by conventional means. Housing 750 and collection canister 708 may be releasably coupled. Mechanisms for selective coupling and decoupling of housing 750 and collection canister 708 include fasteners, latches, clips, straps, bayonet mounts, magnetic couplings, and other devices within the purview of those skilled in the art.

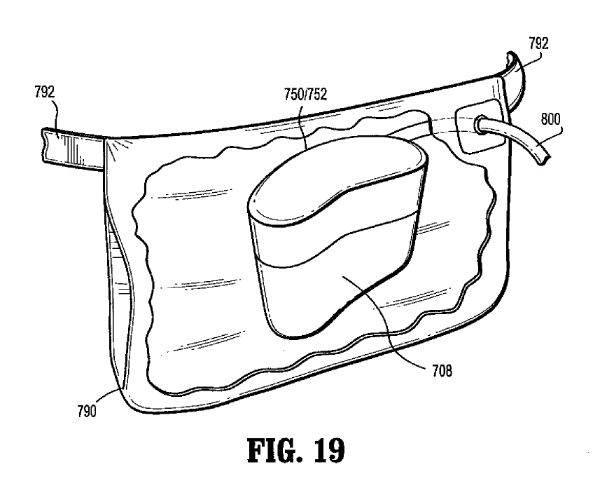

FIG. 19 illustrates a self contained and portable unit where housing 750, 752 control unit and collection canister 708 of wound therapy system 700 are integrally formed or connected as a single assembly for donning by the subject. This assembly is disclosed in commonly assigned U.S. patent application Ser. No. 11/904,411, filed Sep. 27, 2007, the entire contents of which disclosure are hereby incorporated herein by reference. This assembly may be positioned within a support bag or pack 790 having straps 792 which are secured about the subject's waist or shoulder.

While the disclosure has been illustrated and described, it is not intended to be limited to the details shown, since various modifications and substitutions can be made without departing in any way from the spirit of the present disclosure. As such, further modifications and equivalents of the disclosure can occur to persons skilled in the art, and all such modifications and equivalents are intended to be within the spirit and scope of the disclosure as defined by the following claims.

What is claimed is:

1. A negative pressure wound therapy apparatus comprising:
a wound dressing for defining a reservoir over a wound in which a negative pressure may be maintained by forming a substantially fluid-tight seal around the wound;
a fluid conduit in fluid communication with the reservoir and defining a fluid flow path for carrying fluids from the reservoir;
a vacuum source in fluid communication with the fluid conduit, the vacuum source suitable for providing an appropriate negative pressure to the reservoir to stimulate healing of the wound; and
a fluid line coupling, in line with the fluid conduit, the fluid line coupling comprising first and second coupling segments adapted for selectable engagement and disengagement, wherein the at least one of the first and second coupling segments is capable of obstructing fluid flow further includes a post having a bias to deform the fluid conduit section coupled thereto upon disengagement from the other of the first and second coupling segments to obstruct fluid flow, and further wherein the other of the first and second coupling segments includes a pin adapted to engage the post when first and second coupling segments are engaged such that the post is moved against the bias to allow the fluid conduit section to assume an un-deformed shape to permit fluid flow there through.

2. The apparatus according to claim 1, wherein the fluid conduit comprises a first fluid conduit section and a second fluid conduit section, the first fluid conduit section coupled to the first coupling segment of the fluid line coupling, and the second fluid conduit section coupled to the second coupling segment of the fluid line coupling such that fluid may flow between the first and second fluid conduit sections when the first and second coupling segments are engaged and fluid flow is obstructed when the first and second coupling segments are disengaged.

3. The apparatus according to claim 2, wherein the at least one of the first and second coupling segments capable of obstructing fluid flow includes a plug having a bias to engage sealing surfaces on the coupling segment upon disengagement from the other of the first and second coupling segments to obstruct fluid flow.

4. The apparatus according to claim 3, wherein the other of the first and second coupling segments includes a pin adapted to engage the plug when first and second coupling segments are engaged such that the plug is moved away from the sealing surfaces against the bias to permit fluid flow between the first and second coupling segments.

5. The apparatus according to claim 2, wherein the at least one of the first and second coupling segments capable of obstructing fluid flow includes a flipper door having a rotational bias to engage sealing surfaces on the coupling segment upon disengagement from the other of the first and second coupling segments to obstruct fluid flow.

6. The apparatus according to claim 2, wherein the at least one of the first and second coupling segments capable of obstructing fluid flow includes a duckbill valve.

7. The apparatus according to claim 1, wherein the fluid line coupling includes a locking mechanism adapted to move the fluid line coupling between a locked configuration wherein the first and second coupling segments resist separation and an un-locked configuration wherein the first and second mating may be separated.

8. The apparatus according to claim 7, wherein the locking mechanism comprises a locking ring adapted to move interfering members on the first coupling segment between a radially inward position to capture a protrusion on the second coupling segment and radially

9. The apparatus according to claim 1, wherein at least one of the first and second coupling segments comprises a vacuum port including a flange adapted to facilitate connection of the at least one the first and second coupling segments to a wound dressing.

10. The apparatus according to claim 1, wherein at least one of the first and second coupling segments includes a cantilevered locking tab having a locking feature for engaging a corresponding locking feature on the other of the first and second coupling segments, the locking feature positioned on the locking tab between a control surface at a free end of the locking tab and a cantilevered support end of the locking tab.

11. The apparatus according to claim 10, wherein the first coupling segment comprises at least one opening therein, and the second coupling segment comprises a locking tab having a free end configured to protrude laterally through the opening in the first coupling segment to an exterior of the fluid line coupling, the locking tab being biased to selectively maintain the first and second coupling segments in a locked configuration.

12. The apparatus according to claim 11, wherein the first coupling segment comprises a pair of opposed openings therein, and the second coupling segment comprises a pair of opposed locking tabs.

13. The apparatus according to claim 12, wherein the first and second coupling segments are each tapered such that a central region of the fluid line coupling has an expanded width in relation to conduit receiving portions at opposite ends of the coupling segments, and the locking tabs extend substantially across the expanded width.

14. The apparatus according to claim 1 wherein the fluid conduit includes a first circumferential segment and a second circumferential segment, the first circumferential segment having one of translucent or opaque characteristics to prevent direct visualization of flow of exudates through the fluid conduit.

15. The apparatus according to claim 14, wherein the second circumferential segment has transparent characteristics to permit visualization of the flow of exudates through the fluid conduit.

16. The apparatus according to claim 15, wherein the fluid conduit defines a conduit section of a predefined length, the first circumferential segment and the second circumferential segment being incorporated within the conduit section.

17. The apparatus according to claim 15, wherein the conduit section includes a first pair of circumferential segments and a second pair of circumferential segments, the first pair of circumferential segments having one of translucent or opaque characteristics.

18. The apparatus according to claim 17, wherein the circumferential segments of the first pair are in substantial diametrical opposed relation and the circumferential segments of the second pair are in diametrical opposed relation.

19. The apparatus according to claim 3, wherein the collection canister includes at least a wall segment having one of translucent or opaque characteristics to prevent direct visualization through the wall segment of exudates collected within the collection canister and a second wall segment having transparent characteristics to permit direct visualization through the second wall segment of exudates collected within the collection canister.
